(19) United States
(12) Patent Application Publication
(10) Pub. No.: US 2017/0107474 A1
(43) Pub. Date: Apr. 20, 2017

(54) Apparatus, Systems and Methods for Oxygenating Liquid Media to Enhance Microorganism Growth

(71) Applicants: Sean FARMER, Miami, FL (US); Sharmistha MAZUMDER, Copley, OH (US); Xiao-Zhou ZHANG, Solon, OH (US); Maja MILOVANOVIC, North Royalton, OH (US)

(72) Inventors: Sean FARMER, Miami, FL (US); Sharmistha MAZUMDER, Copley, OH (US); Xiao-Zhou ZHANG, Solon, OH (US); Maja MILOVANOVIC, North Royalton, OH (US)

(21) Appl. No.: 15/297,491

(22) Filed: Oct. 19, 2016

Related U.S. Application Data
(60) Provisional application No. 62/243,721, filed on Oct. 20, 2015.

(51) Int. Cl. C12M 1/34; C12M 1/36; C12M 1/02; C12M 1/00

(52) U.S. Cl. CPC C12M 41/34; C12M 29/18; C12M 29/14; C12M 29/20; C12M 29/06; C12M 41/48; C12M 27/00

(57) ABSTRACT

A bioreactor includes a fermentation tank and an external loop passage having a first end connected to an inlet port of the fermentation tank and a second end connected to an outlet port of the fermentation tank. A pressurized gas source is in fluid communication with at least one oxygenation port connected with the external loop passage, to supply pressurized gas to a liquid medium passing through the external loop passage, wherein the pressurized gas is sufficient to dissolve oxygen into the liquid medium and to force the liquid medium into the fermentation tank through the inlet port. A vent port is connected with a top portion of the fermentation tank for exhausting pressurized gas from the fermentation tank.

### CROSS-REFERENCE TO RELATED APPLICATION

The present application claims priority to and all benefit of U.S. Provisional Patent Application Ser. No. 62/243,721, filed on Oct. 20, 2015, for APPARATUS, SYSTEMS AND METHODS FOR OXYGENATING LIQUID MEDIA TO ENHANCE BACTERIA GROWTH, the entire disclosure of which is fully incorporated herein by reference.

### BACKGROUND

The use of live bacteria and other microorganisms has been successfully incorporated into a wide variety of applications, including, for example, waste remediation, enhanced oil recovery, agricultural control agents, pesticides, and mining. In most such applications, a high density of live microorganisms is desirable to maximize effectiveness and to minimize the amount of material to be transported and deployed for treatment. To this end, a bioreactor or fermentor is typically provided with controlled amounts of starter bacteria, water, nutrients, pH control agents, and defoaming agents to provide a favorable environment for supporting rapid bacterial growth. For aerobic (i.e. oxygen dependent) fermentation, oxygen is also supplied to the fermentation medium to support rapid cell growth.

### SUMMARY

The present application contemplates systems and methods for oxygenating liquid media in a batch process bioreactor to maximize bacterial growth, while minimizing fluid shear and other conditions that are harmful to the live bacteria. The dissolved oxygen content, oxygen transfer rate, and oxygen uptake rate are factors which determine the effectiveness of the fermentation systems. These may be controlled by adjusting the flow rates, agitation, and oxygen flow paths.

In an exemplary embodiment, a bioreactor includes a fermentation tank and an external loop passage having a first end connected to an inlet port of the fermentation tank and a second end connected to an outlet port of the fermentation tank. A pressurized gas source is in fluid communication with at least one oxygenation port connected with the external loop passage, to supply pressurized gas to a liquid medium passing through the external loop passage, wherein the pressurized gas is sufficient to dissolve oxygen into the liquid medium and to force the liquid medium into the fermentation tank through the inlet port. A vent port is connected with a top portion of the fermentation tank for exhausting pressurized gas from the fermentation tank.

In another exemplary embodiment, a bioreactor includes a fermentation tank having an inlet port and an outlet port, first and second oxygenating mechanisms each assembled with the fermentation tank and configured to supply oxygenating bubbles to a fluid disposed in the fermentation tank, and a controller in electronic communication with the first and second oxygenating mechanisms to automatically control operation of the first and second oxygenating mechanisms to adjust an oxygen transfer rate during a fermentation process in accordance with dissolved oxygen parameters stored in the program logic controller, to correspond with an increasing oxygen demand during the fermentation process.

### BRIEF DESCRIPTION OF THE DRAWINGS

These and other features and advantages of the present invention will become better understood with regard to the following description and accompanying drawings in which:

FIG. 1 is a schematic side cross-sectional view of a bioreactor, according to an exemplary embodiment;

FIG. 2 is a schematic side cross-sectional view of another bioreactor, according to an exemplary embodiment;

FIG. 2A is a side cross-sectional view of another bioreactor, according to an exemplary embodiment;

FIG. 3 is a side schematic side cross-sectional view of another bioreactor, according to another exemplary embodiment;

FIGS. 3A-3D are front views of exemplary diffuser discs for use with an air lift passage of a bioreactor;

FIG. 3E is a side view of an elbow portion of an external loop for a bioreactor, according to an exemplary embodiment; and

FIG. 4 is a side schematic side cross-sectional view of another bioreactor, according to another exemplary embodiment.

### DETAILED DESCRIPTION

The Detailed Description merely describes exemplary embodiments of the invention and is not intended to limit the scope of the claims in any way. Indeed, the invention is broader than and unlimited by the exemplary embodiments, and the terms used in the claims have their full ordinary meaning.

Air or oxygen supplied to a fermentation chamber of a bioreactor for aerobic fermentation may be introduced as bubbles of air or oxygen near the bottom of the fermentation chamber, which functions to increase oxygen transfer to the liquid medium as the bubbles pass upward through the contents of the chamber, and also to agitate the contents of the chamber, for example, in addition to or in place of impeller blades or other mechanical agitation mechanisms. In some applications, air bubble agitation of the fermentation medium may be preferable to mechanical agitation mechanisms that may impart undesirable shear forces on the live bacteria within the liquid medium (which may kill some of the bacteria). However, the limited amount of oxygen in air (about 21%) and the limited solubility of oxygen into water present challenges for providing sufficient oxygen to a fermentation system for rapid bacteria growth from internal air bubble agitation alone, particularly during the more advanced stages of cell proliferation where substantial, increasing amounts of oxygen are needed.

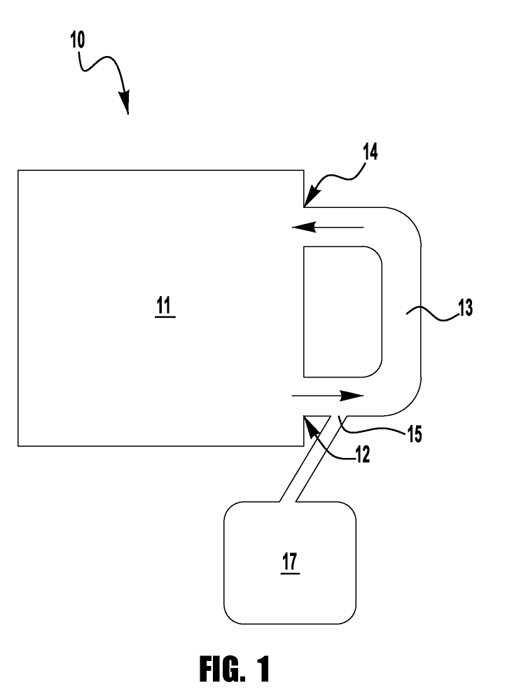

According to an exemplary aspect of the present application, a batch-type bioreactor may be provided with an external passage for recirculating and oxygenating a portion of the liquid medium while minimizing mechanical agitation of the medium. In one such exemplary bioreactor 10, as schematically shown in FIG. 1, an oxygen-poor portion of the liquid medium is expelled through an outlet port 12 in the bioreactor's fermentation tank 11 into an external loop passage 13. Pressurized air (or any other suitable oxygen containing gas) is supplied from a pressurized gas source 17 to the loop passage through one or more oxygenation ports 15 to oxygenate the liquid medium as the liquid medium passes through the loop passage 13. The liquid medium is returned to the fermentation tank 11 through an inlet port 14 in the fermentation tank. By the time the expelled liquid medium reaches the inlet port 14, the liquid medium has been oxygenated by the pressurized gas in the external loop passage 13, and the oxygen-rich liquid medium is reintroduced to the fermentation tank 11 to support optimal growth of the bacteria.

While the expelled liquid medium may be pumped through the external loop passage by a fluid recirculating pump connected with the passage, in another embodiment, the pressurized oxygenating gas supplied to the external loop passage is sufficient to draw the liquid medium from the fermentation tank and to force the liquid medium through the external loop passage to the inlet port. This pressurized gas forced circulation of the liquid medium eliminates the challenges of contamination, filtration, and/or cleaning of an in-line pump.

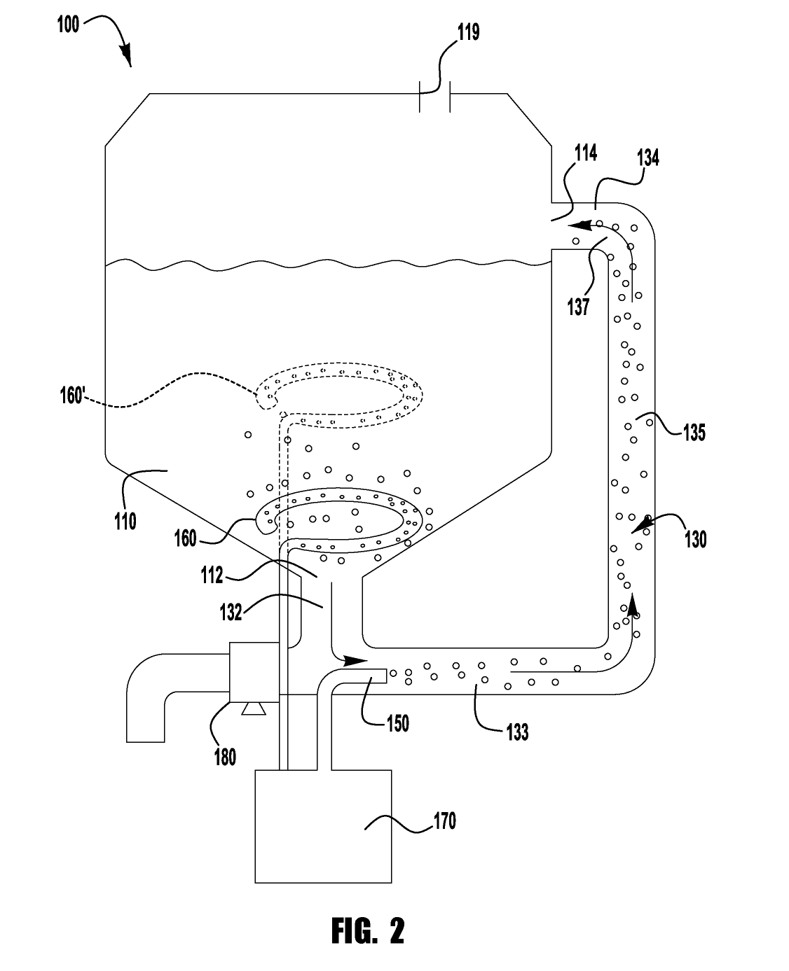

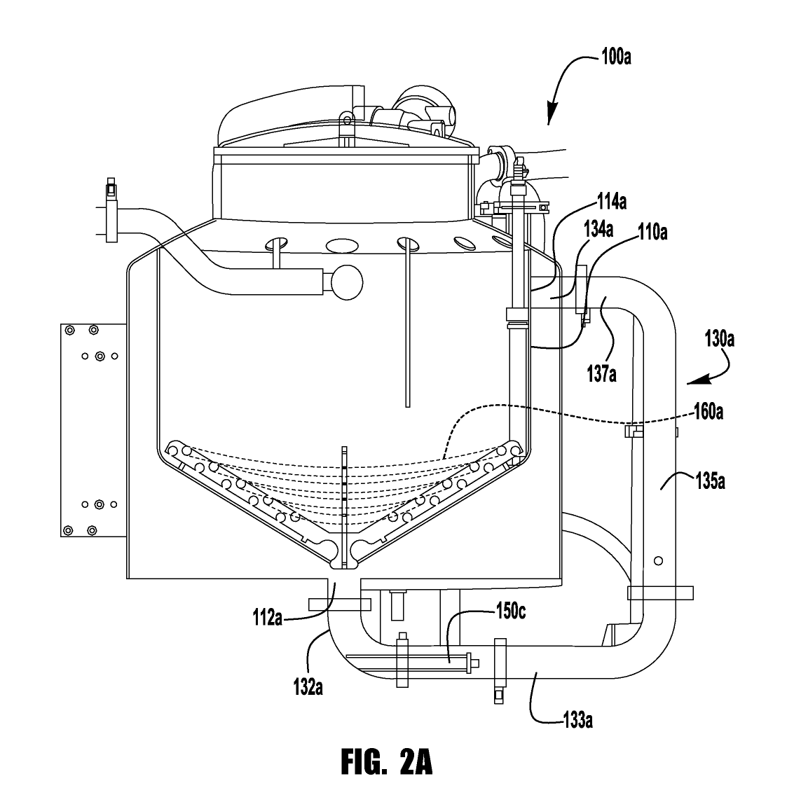

The outlet and inlet liquid medium ports may be provided on the fermentation tank in a variety of locations and orientations. In one embodiment, as shown in FIG. 2, a bioreactor 100 includes a fermentation tank 110 and an external loop passage 130 (formed from tubing or some other suitable conduit) having a first end 132 connected with an outlet port 112 disposed at a bottom end of the fermentation tank 110, and a second end 132 connected with an inlet port 114 disposed on a side wall of the fermentation tank. The outlet port may also be connected with a drain valve 180 for supplying liquid medium with active bacteria to a test site or use site. The liquid medium is expelled through the outlet port and into the external loop passage by gravity flow, and at least one oxygenation port 150 is connected with a lower leg 133 of the external loop passage to supply pressurized gas from a pressurized gas source 170 to the lower leg. The pressurized gas forces the expelled fluid up a vertical portion 135 of the external loop passage 130 and through an upper leg 137 of the external loop passage to the inlet port 114 for reintroduction of the fluid into the fermentation tank. While the inlet port may be positioned in a variety of locations on the side of the fermentation tank, in one embodiment, the inlet port is be positioned above the upper surface of the liquid medium in the fermentation tank, for example, to reduce the pressure required to pump the fluid back into the tank. Pressurized gas flowing into the fermentation tank 110 through the inlet port 114 may be exhausted from the tank through a vent port 119 in an upper portion of the tank. The turbulence resulting from circulation of the liquid medium through the external loop passage may also facilitate release of carbon dioxide from the fluid, which may also be exhausted through the vent port 119.

As the fluid is forced through the external loop passage, oxygen from the pressurized gas is dissolved into the fluid to provide an oxygen rich medium for bacterial growth. In one embodiment, the resulting reduced density of the oxygenated fluid is sufficient to generate circulation of the fluid upward through the external loop passage. In other embodiments, the pressurized gas supplied to the lower leg of the external loop passage is sufficient to force the fluid upward toward the lower pressure upper (or headspace) portion of the fermentation tank, and provides for more rapid circulation of the fluid as compared to air sparger generated differential bulk density fluid circulation. In an exemplary embodiment, the external loop passage is pressurized to 0.1 to 40 psig air at a flow rate of 5 to 45 cubic feet per minute, to support a liquid medium flow rate of 1 to 95 gallons per minute.

According to another exemplary aspect of the present application, a bioreactor with an external loop passage oxygenation arrangement, as described above, may additionally be provided with a “micro bubbler” aerator or air sparger 160 at a base portion of the fermentation tank 110, to further oxygenate and circulate the fluid within the tank. The formation of these micro bubbles within the fermentation tank further support the growth of high oxygen demand microbes, as lack of oxygen is a rate limiting step in the fermentation process. The air sparger is connected with a pressurized gas source to generate very small air bubbles at lower pressures (e.g., 2 to 5 psig) at the base of the fermentation tank. While many different types of air spargers may be used, in one embodiment, an air sparger is formed from a flexible air tube having very small holes or perforations (e.g., 2000 to 3500 slits per meter) to produce very small bubbles that are more easily dissolved in the liquid medium (as compared to larger bubbles).

FIG. 2A illustrates an exemplary bioreactor 100a includes a fermentation tank 110a and an external loop passage 130a (formed from clamped sections of tubing) having a first end 132a connected with an outlet or drain port 112a disposed at a bottom end of the fermentation tank 110a, and a second end 134a connected with an inlet port 114a disposed on a side wall of the fermentation tank. The liquid medium is expelled through the outlet port and into the external loop passage by gravity flow, and at least one oxygenation port 150a is connected with a lower leg 133a of the external loop passage to supply pressurized gas from a pressurized gas source (not shown) to the lower leg. The pressurized gas forces the expelled fluid up a vertical portion 135a of the external loop passage 130a and through an upper leg 137a of the external loop passage 130a to the inlet port 112a for reintroduction of the fluid into the fermentation tank. Pressurized gas flowing into the fermentation tank 110a through the inlet port 114a may be exhausted from the tank through a vent port in an upper portion of the tank. The bioreactor includes a “micro bubbler” aerator or air sparger 160a at a base portion of the fermentation tank 110a, to further oxygenate and circulate the fluid within the tank.

According to another aspect of the present application, to oxygenate the liquid medium in a fermentation tank more effectively and efficiently using both external loop gas pressurized oxygenation and internal air sparger oxygenation, the air sparger may be positioned to produce aerating bubbles in the fermentation tank at a location that is vertically spaced apart from the liquid medium outlet port, to facilitate expulsion of relatively oxygen poor liquid medium into the external loop passage while retaining the relatively oxygen rich (as oxygenated by the air sparger) liquid medium in the fermentation tank. For example, the air sparger may be vertically proximal to the inlet port and vertically distal to the outlet port. This may be accomplished by positioning the air sparger at a vertically elevated position with respect to the outlet port, or by positioning the outlet port at a vertically elevated position with respect to the air sparger.

In one embodiment, as shown in phantom in FIG. 2, an air sparger 160′ may be positioned above the base portion of the fermentation tank 110, spaced apart from the outlet port 112, for example, near a midpoint of the liquid medium filled portion of the fermentation tank. The bubbles generated by the air sparger 160′ float upward in the liquid medium to oxygenate an upper portion of the liquid medium within the fermentation tank. The outlet port to the external loop passage, positioned at the bottom portion of the fermentation tank, expels a lower portion of the liquid medium, substantially untreated by the air sparger, into the external loop passage for external loop pressurized oxygenation.

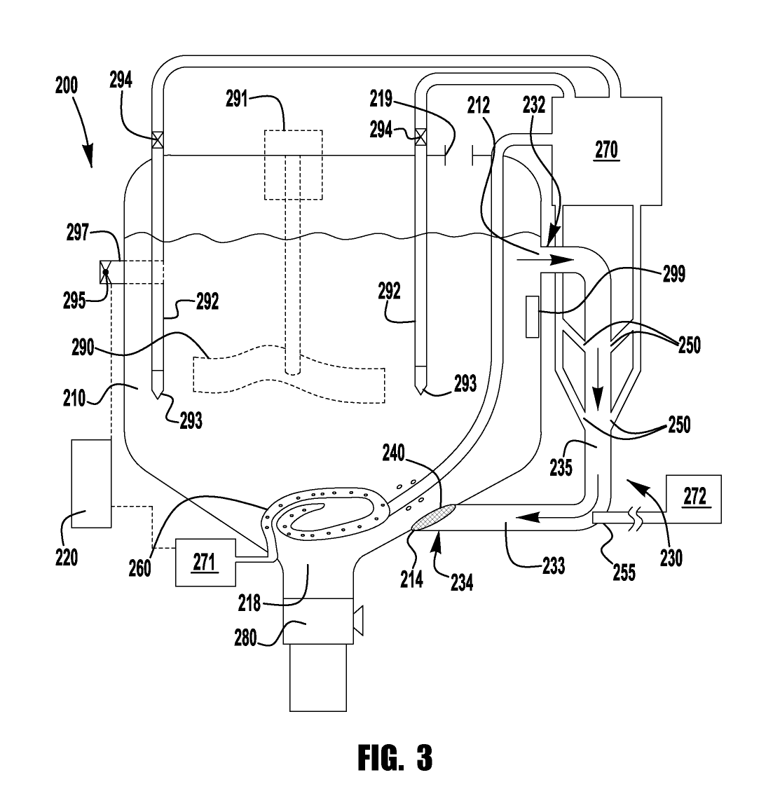

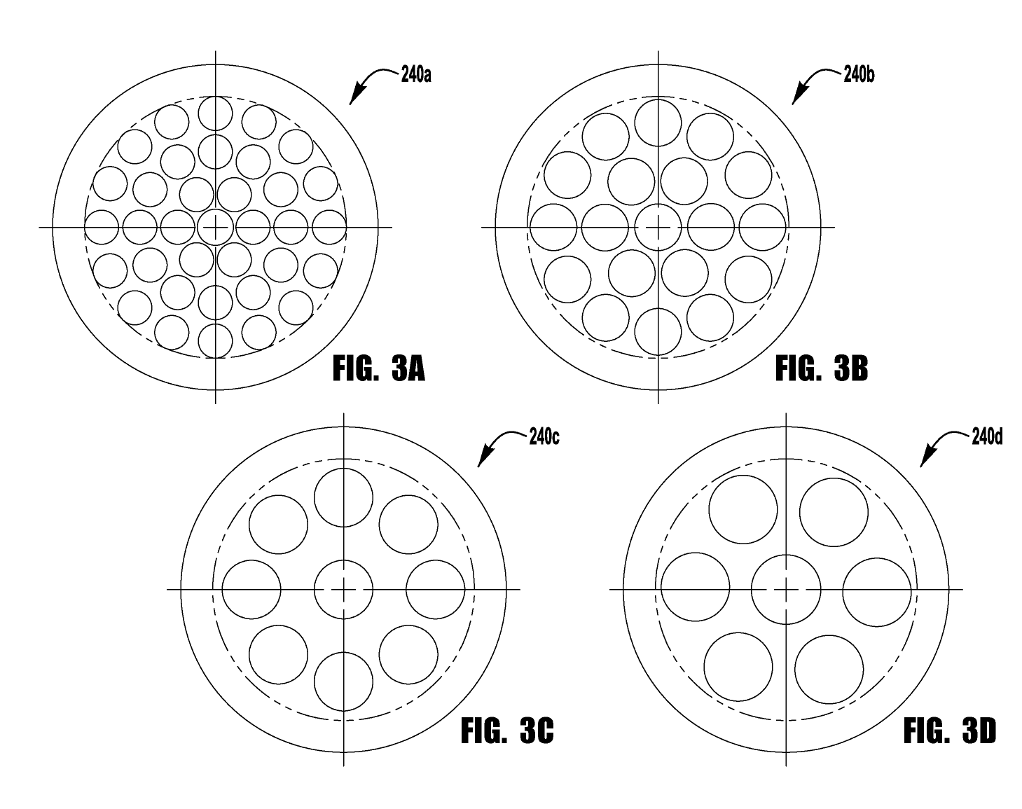

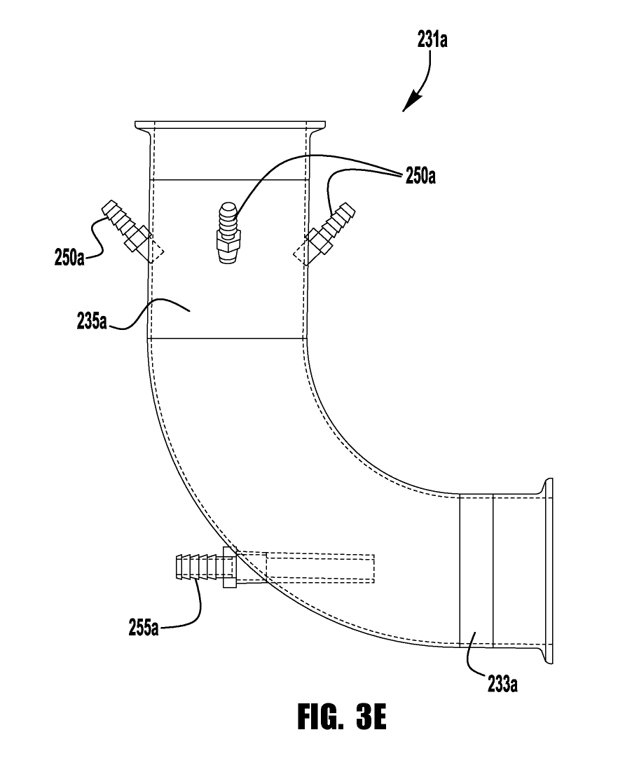

In another embodiment, as shown in FIG. 3, a bioreactor 200 includes a fermentation tank 210 and an external loop passage 230 (formed from tubing or some other suitable conduit) having a first end 232 connected with an outlet port 212 disposed on a side wall of the fermentation tank, near an upper surface of the liquid medium, and a second end 234 connected with an inlet port 214 disposed at a bottom portion of the fermentation tank. As shown, the inlet port 214 may be disposed on a side wall (e.g., the conical bottom side wall) at the bottom portion of the fermentation tank to facilitate circulation of fluids reintroduced into the tank. In such an embodiment, a separate drain port 218 may be provided for connection with a drain valve 280, for supplying liquid medium with active bacteria to a test site or use site. One or more oxygenation ports 250 connected with a vertical portion 235 of the external loop passage 230 are angled downward to direct pressurized gas (supplied from a pressurized gas source 270) through the external loop passage toward the inlet port, to pull liquid medium from the fermentation tank through the outlet port and into the external loop passage 230. Additionally or alternatively, one or more oxygenation ports 255 connected with a lower leg portion 233 of the external loop passage 230 are oriented horizontally to direct pressurized gas (supplied from a pressurized gas source 270) through the lower leg portion toward the inlet port, to pull liquid medium from the fermentation tank through the outlet port and into the external loop passage 230. The pressurized gas forces the expelled fluid downward through the vertical portion 235 of the external loop passage and through the lower leg 233 of the external loop passage to the inlet port for reintroduction of the fluid into the fermentation tank. Pressurized gas flowing into the fermentation tank 210 through the bottom end inlet port 214 may pass upward through the fermentation tank to further oxygenate the liquid medium within the fermentation tank, and may be vented through a vent port 219 in an upper portion of the tank 210.

A perforated or diffuser screen or plate 240 may be positioned in or adjacent to the inlet port to produce smaller bubbles of the pressurized gas, to facilitate more effective dissolution of oxygen into the liquid medium within the fermentation tank, thereby enhancing dissolved oxygen levels. The apertures in the diffuser plate may be sized and positioned to accommodate the desired flow rate through the external passage. Exemplary diffuser plates 240a, 240b, 240c, 240d are shown in FIGS. 3A-3D. Additionally or alternatively, one or more diffuser screens or plates may be positioned within the external loop passage, downstream of the oxygenation port, in any of the embodiments described herein, to facilitate more effective dissolution of oxygen into the liquid medium within the external loop passage, thereby enhancing dissolved oxygen levels.

Many different arrangements may be provided for the oxygenation ports of the external loop passage having an upper outlet port and a lower inlet port, as shown schematically in FIG. 3. In an exemplary embodiment, as shown in FIG. 3E, a lower elbow 231a of an external loop passage includes four upper oxygenation ports 250a disposed uniformly (i.e., at ninety degree intervals) around a vertical portion 235a of the elbow 231a, and one lower oxygenation port 255a connected with a horizontal or lower leg portion 233a of the elbow 231a. The upper oxygenation ports 250a are angled downward (e.g., at a 45° angle) to direct pressurized gas through the external loop passage toward the inlet port, to pull liquid medium from the fermentation tank through the outlet port and into the external loop passage 230. The lower oxygenation port 255 is oriented horizontally to direct pressurized gas through the lower leg portion 233a toward the inlet port.

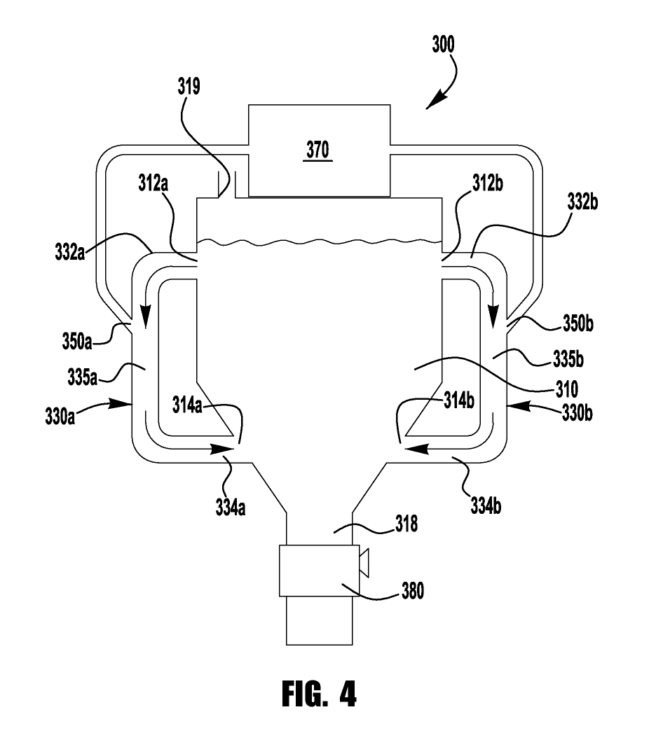

Additional oxygenating mechanisms may also be provided with the bioreactor, as shown in the embodiment of FIG. 3, but also applicable to the other embodiments described herein, including the embodiments of FIGS. 2 and 4. An air sparger or microbubbler 260, as described above, may be provided (e.g., at the bottom portion of the fermentation tank) to provide additional oxygenating gas bubbles. Additionally or alternatively, drop-down nozzle ended tubes or hollow rods 292 may extend down into the tank to a predetermined depth, to supply air jets for further oxygenation of the fermentation fluid. The nozzles 293 may be any suitable nozzle ends, including, for example, a ⅛ inch or ¼ inch air nozzle or high velocity air jet, manufactured by STREAMTEK. Manually or electronically operated valves 294 may be provided to selectively control air jet oxygenation of the fermentation tank 210. As another example, an impeller 290 may additionally or alternatively be provided to agitate the liquid media, thereby facilitating dissolution of oxygen in the liquid media. Many different types of impellers may be used, including, for example, a vortex impeller configured to create a vortex in the fluid, independently or in combination with the external air loop oxygenation arrangements described above. A pump or valve 295 and supply port 297 may additionally or alternatively be provided in fluid communication with the fermentation tank 210 to supply oxygen generating or oxygen dissolving chemicals to increase the dissolved oxygen content of the liquid media. Similar impeller and chemical oxygenating arrangements may also be used in an upward directed external loop oxygenation system, as described in the exemplary embodiment of FIG. 2.

During the preparation of a batch of microbiological material, the amount of dissolved oxygen in the liquid media needed for successful bacteria growth may increase dramatically during the course of the fermentation process. According to another aspect of the present application, a fermentation system may be configured to adjust the oxygenation operations of the fermentation system based on the amount of oxygen needed for bacterial growth at any point during the bacterial growth process. This adjustment may involve program logic controller (PLC) controlled selective use or variance of one or more oxygenating mechanisms, including for example, the downward directed external loop aeration described above (with varying air jet flow rates), the upward directed external loop aeration described above (with varying air jet flow rates), the perforated tube air sparger described above (with varying air flow rates), one or more impellers within the fermentation tank (at varying rotational speeds, calculated to facilitate enhanced oxygenation while minimizing shear damage to the live bacteria), and/or a pump or valve supplying oxygen generating or oxygen dissolving chemicals through a supply port (in varying specified amounts). In one such embodiment, as shown in FIG. 3, a controller (e.g., program logic controller or PLC), shown schematically at 220, may be in electronic communication with one or more of an adjustable air source 271 for an air sparger 260, adjustable air sources 270, 272 for external loop oxygenation ports 250, 255, control valves 294 for drop-down air jet nozzle ended tubes 292, a driving mechanism 291 (e.g., motor) for one or more adjustable speed impellers 290, and a pump/valve 295 for a oxygenating chemical supply port 297. By controlling use and settings of one or more of the system's oxygenating mechanisms, an oxygen transfer rate may be adjusted (e.g., oxygen transfer rates between about 100 and about 500 millimoles of oxygen per liter per hour) to achieve a desired amount of dissolved oxygen in the fermentation fluid (e.g., between about 2% and about 30% dissolved oxygen).

In an exemplary implementation of a fermentation system (as shown in the exemplary embodiment of FIG. 3, but also applicable to the other embodiments described herein, including the embodiments of FIGS. 2 and 4), an early, first stage of fermentation involves PLC controlled supply of low pressure air (from air source 271) to the air sparger 260, and PLC controlled supply of air to at least one of the horizontal oxygenation port 255 (from air source 272) and downward angled oxygenation ports 250 (from air source 272), sufficient to maintain fluid circulation through the external loop passage 230. In a second stage of fermentation (where oxygen demands have increased), the PLC 220 may control air sources 271, 270, 272 to supply increased air pressure to the air sparger 260 and to one or more of the oxygenation ports 250, 255 to supply additional oxygen to the liquid media. In a third stage of fermentation (where oxygen demands have increased further), the PLC 220 may control the impeller 290 to agitate the fluid for increased dissolution of oxygen in the liquid media, and/or an oxygen generating or oxygen dissolving chemical may be supplied to the fermentation tank through the valve 295 and supply port 297 to facilitate dissolution of oxygen in the liquid media. Within these preset stages, the oxygenation mechanisms may be variably controlled (e.g., varying air pressure to air spargers, air jets, and/or external loop oxygenation ports, and/or varying impeller speed). This variable use of the bioreactor's oxygenating mechanisms may be effected in accordance with a preset time-based microbiological material specific recipe stored by the controller 220, based on the specific oxygen demands, over time and at different stages of microbiological growth. Additionally or alternatively, a preset microbiological material specific recipe may specifically identify the amount of required dissolved oxygen (e.g., dissolved oxygen levels between about 2% and about 30%) over the duration of the fermentation process, and the bioreactor system may receive dissolved oxygen data from a dissolved oxygen (DO) sensor 299 in the fermentation tank 210, making corresponding adjustments to use and settings of the oxygenation mechanisms in response to these DO sensor readings.

In still other embodiments, a bioreactor may be provided with multiple (i.e., two or more) external loop passages to provide for increased fluid circulation and oxygenation. FIG. 4 schematically illustrates a bioreactor 300 including a fermentation tank 310 and two external loop passages 330a, 330b (formed from tubing or some other suitable conduit) each having a first end 332a, 332b connected with an outlet port 312a, 312b disposed on a side wall of the fermentation tank, near an upper surface of the liquid medium, and a second end 334a, 334b connected with an inlet port 314a, 314b disposed at a bottom portion of the fermentation tank 310. A separate drain port 318 may be provided for connection with a drain valve 380, for supplying liquid medium with active bacteria to a test site or use site. One or more oxygenation ports 350a, 350b connected with a vertical portion 335a, 335b of the external loop passages 330a, 330b are directed downward to direct pressurized gas (supplied from a pressurized gas source 370) through the external loop passage toward inlet portion, to pull liquid medium from the fermentation tank through the outlet ports 312a, 312b and into the external loop passages 330a, 330b. The pressurized gas forces the expelled fluid downward through the vertical portion 335a, 335b of the external loop passages and through lower leg 333a, 333b of the external loop passages to the inlet ports for reintroduction of the fluid into the fermentation tank. Pressurized gas flowing into the fermentation tank 310 through the bottom end inlet ports 314a, 314b may pass upward through the fermentation tank to further oxygenate the liquid medium within the fermentation tank, and may be vented through a vent port 319 in an upper portion of the tank 310.

In other embodiments (not shown), a bioreactor may include multiple external loop passages with oxygenation ports arranged to direct recirculating fluid upward through the external loop passages, similar to the arrangement of FIG. 2. In still other embodiments (not shown), a bioreactor including multiple external loop passages may include at least one external loop passage configured for downward flow of recirculating fluid, and at least one external loop passage configured for upward flow of recirculating fluid, as described in greater detail in the above embodiments.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

We claim:

1. A bioreactor comprising:
a fermentation tank having an inlet port and an outlet port;
an external loop passage having a first end connected to the inlet port and a second end connected to the outlet port;
a pressurized gas source;
at least one oxygenation port in fluid communication with the pressurized gas source and connected with the external loop passage to supply pressurized gas to a liquid medium passing through the external loop passage, wherein the pressurized gas is sufficient to force the liquid medium into the fermentation tank through the inlet port; and
a vent port connected with a top portion of the fermentation tank for exhausting pressurized gas from the fermentation tank.

2. The bioreactor of claim 1, wherein the outlet port is disposed in an upper portion of the fermentation tank and the inlet port is disposed in a lower portion of the fermentation tank.

3. The bioreactor of claim 1, wherein the outlet port is disposed in a lower portion of the fermentation tank and the inlet port is disposed in an upper portion of the fermentation tank.

4. The bioreactor of claim 1, further comprising an air sparger disposed in the fermentation tank.

5. The bioreactor of claim 4, wherein the air sparger is vertically proximal to the inlet port and vertically distal to the outlet port.

6. The bioreactor of claim 1, wherein the at least one oxygenation port is connected with a lower leg portion of the external loop passage.

7. The bioreactor of claim 1, wherein the at least one oxygenation port is connected with a vertical portion of the external loop passage.

8. The bioreactor of claim 1, further comprising a program logic controller configured to control at least one oxygenating mechanism of the bioreactor to control an oxygen transfer rate in accordance with dissolved oxygen parameters stored in the program logic controller, the at least one oxygenating mechanism including the supply of pressurized gas to the external loop passage.

9. The bioreactor of claim 8, wherein the program logic controller is configured to control the at least one oxygenating mechanism to maintain an oxygen transfer rate between about 100 and about 500 millimoles of oxygen per liter per hour.

10. The bioreactor of claim 8, wherein the program logic controller is configured to control the at least one oxygenating mechanism to maintain a dissolved oxygen content between about 2% and about 30%.

11. The bioreactor of claim 8, wherein the at least one oxygenating mechanism further includes a pressurized air sparger disposed in the fermentation tank.

12. The bioreactor of claim 11, wherein the air sparger comprises a microbubbler.

13. The bioreactor of claim 8, wherein the at least one oxygenating mechanism further includes an impeller disposed in the fermentation tank.

14. The bioreactor of claim 13, wherein the impeller comprises a vortex impeller.

15. The bioreactor of claim 8, wherein the at least one oxygenating mechanism further includes an oxygenating chemical supply valve or pump, in fluid communication with the fermentation tank.

16. The bioreactor of claim 1, further comprising a diffuser plate disposed in the inlet port of the fermentation tank, the diffuser plate including a pattern of apertures sized and positioned to enhance bubble formation and increase dissolved oxygen levels while permitting a desired fluid flow rate through the external loop passage.

17. A bioreactor comprising:
a fermentation tank having an inlet port and an outlet port;
first and second oxygenating mechanisms each assembled with the fermentation tank and configured to supply oxygenating bubbles to a fluid disposed in the fermentation tank; and
a controller in electronic communication with the first and second oxygenating mechanisms to automatically control operation of the first and second oxygenating mechanisms to adjust an oxygen transfer rate during a fermentation process in accordance with dissolved oxygen parameters stored in the program logic controller, to correspond with an increasing oxygen demand during the fermentation process.

18. The bioreactor of claim 17, wherein the first oxygenating mechanism comprises an external loop passage having a first end connected to the inlet port and a second end connected to the outlet port, a pressurized gas source controlled by the controller, and at least one oxygenation port in fluid communication with the pressurized gas source and connected with the external loop passage to supply pressurized gas to a liquid medium passing through the external loop passage, wherein the pressurized gas is sufficient to force the liquid medium into the fermentation tank through the inlet port.

19. The bioreactor of claim 18, wherein the second oxygenating mechanism comprises at least one of an air sparger, an impeller, an air jet nozzle ended tube, and an oxygenating chemical supply valve or pump.

20. The bioreactor of claim 17, further comprising a dissolved oxygen sensor in communication with the controller, the controller being configured to adjust at least one of the first and second oxygenating mechanisms in response to dissolved oxygen parameters measured by the dissolved oxygen sensor and communicated to the controller.
